(19) United States
(12) Patent Application Publication
(10) Pub. No.: US 2026/0248541 A1
(43) Pub. Date: Aug. 27, 2026

(54) Guide Templates For Percutaneous Inserter System, And Related Instrumentation, Assemblies, And Methods

(71) Applicant: DePuy Synthes Products, Inc., Raynham, MA (US)

(72) Inventors: Joel Oberli, Niederdorf (CH); This Aebi, Grenchen (CH)

(21) Appl. No.: 19/062,188

(22) Filed: Feb. 25, 2025

(51) Int. Cl. A61B 17/80; A61B 17/17; A61B 90/00

(52) U.S. Cl. CPC A61B 17/8014; A61B 17/1728; A61B 90/08; A61B 90/39; A61B 2090/0811; A61B 2090/3937

(57) ABSTRACT

A template for guiding bone screw insertion through soft tissue and through holes in an underlying, twisted bone plate includes: first and second ends longitudinally opposite each other; first and second sides laterally opposite each other; outer and bone-facing surfaces opposite each other; and aperture arrangements longitudinally spaced from each other. At least some of the aperture arrangements each include: one or more apertures that are longitudinally elongated and extend from the outer surface to the bone-facing surface; and a marker indicating a location of an underlying plate hole. The one or more apertures of the foregoing at least some of the aperture arrangements define a maximum total lateral dimension. The maximum total lateral dimension increases from at least one of the aperture arrangements to a subsequent one of the aperture arrangements in a forward longitudinal direction along the template toward the second end.

### TECHNICAL FIELD

The present invention relates to bone fixation, particularly a percutaneous inserter system for guiding bone fixation members to a target location through an associated fixation hole in a bone plate, and related instrumentation, assemblies, and methods.

### BACKGROUND

Bone plate systems for the internal fixation of bone fractures are well known. Conventional bone plate systems are particularly well-suited to promote the healing of a fracture. A bone fixation member, such as a bone screw, is inserted through a fixation aperture or hole in a bone plate and is threaded into bone to compress, neutralize, buttress, tension, band, and/or bridge the fracture ends together. Cannulas can be employed with the bone plating system to facilitate insertion of the bone fixation member into the bone while reducing contact with the soft tissue through which the bone fixation member must pass. Guide templates can be employed for placement on the outer surface of the skin to guide the position of the cannulas for targeting fixation holes of the bone plate.

### SUMMARY

According to an embodiment of the present disclosure, a surgical system for bone fixation includes a bone plate for affixation to underlying bone, and a template for placement on an outer surface of soft tissue overlaying the bone plate. The bone plate includes a first plate end and a second plate end opposite each other along a longitudinal plate axis, an outer plate surface and an opposed bone-facing plate surface extending substantially parallel to each other between the first and second plate ends, and a plurality of fixation holes extending from the outer plate surface to the bone-facing plate surface. The plurality of fixation holes are arranged in a longitudinal series that begins adjacent the first plate end and terminates adjacent the second plate end. The bone plate also includes a twisted plate portion along which the outer and bone-facing plate surfaces have respective twisted geometries about the longitudinal plate axis. The template includes first and second template ends opposite each other along a longitudinal direction. The template defines a plurality of apertures that are located between the first and second template ends and are configured to orient insertion trajectories of bone screws through the soft tissue and through associated fixation holes in the bone plate. At least a subset of the plurality of apertures are sized and positioned such that, when the template overlays the bone plate, at least one of the insertion trajectories intersects both of 1) an aperture of the subset of apertures, and 2) an associated fixation hole extending through the twisted plate portion.

According to another embodiment of the present disclosure, a template is configured for guiding bone screw insertion through soft tissue and through holes in an underlying, twisted bone plate. The template includes: first and second ends longitudinally opposite each other; first and second sides laterally opposite each other; outer and bone-facing surfaces opposite each other; and aperture arrangements longitudinally spaced from each other. At least some of the aperture arrangements each include: one or more apertures that are longitudinally elongated and extend from the outer surface to the bone-facing surface; and a marker indicating a location of an underlying plate hole. The one or more apertures of the foregoing at least some of the aperture arrangements define a maximum total lateral dimension. The maximum total lateral dimension increases from at least one of the aperture arrangements to a subsequent one of the aperture arrangements in a forward longitudinal direction along the template toward the second end.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the features of the present application, there is shown in the drawings illustrative embodiments. It should be understood, however, that the application is not limited to the precise arrangements and instrumentalities shown. In the drawings:

FIG. 1A is a perspective view of a surgical system that includes a bone plate and a guide template to facilitate targeting fixation holes of the bone plate, according to an embodiment of the present disclosure;

FIG. 1B is a perspective top view of the bone plate illustrated in FIG. 1A;

FIGS. 1C-1D are sectional end views of the bone plate taken along section planes extending through holes H1 and H16 labeled in FIG. 1B;

FIG. 1E is a perspective side view of the bone plate illustrated in FIG. 1A;

FIG. 1F is a perspective view of respective end regions of the template and bone plate illustrated in FIG. 1A;

FIG. 1G is a sectional perspective view of aspects of the system (including the template and bone plate) illustrated in FIG. 1A;

FIG. 2A is a top plan view of the template illustrated in FIG. 1A;

FIG. 2B is a sectional end view of the template, taken along section line 2B-2B shown in FIG. 2A;

FIGS. 2C-2E are enlarged plan views of respective portions of the template illustrated in FIG. 2A;

FIGS. 3A-3F are perspective views showing stages of using the template illustrated in FIG. 2A to target fixation holes of the bone plate, according to an embodiment of the present disclosure;

FIG. 4A is a top plan view of another template for use with the bone plate illustrated in FIGS. 1A-1G, according to another embodiment of the present disclosure;

FIGS. 4B-4C are enlarged plan views of respective portions of the template illustrated in FIG. 4A;

FIGS. 5A-5B are perspective views showing stages of using the template illustrated in FIG. 4A for preparing incisions at respective locations of the soft tissue for targeting underlying fixation holes of the bone plate;

FIG. 5C is a perspective view of a guide sleeve of the surgical system illustrated in FIG. 1A extending through an incision and in alignment with an underlying fixation hole of the bone plate;

FIG. 6A is a top plan view of another template for use with the bone plate illustrated in FIGS. 1A-1G, according to another embodiment of the present disclosure; and

FIG. 6B is an enlarged plan view of a respective portion of the template illustrated in FIG. 6A.

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this disclosure is not limited to the specific devices, assemblies, systems, methods, applications, conditions or parameters described and/or shown herein, and that the terminology used herein is for the purpose of describing particular embodiments by way of example only and is not intended to be limiting of the scope of the present disclosure.

The embodiments disclosed herein pertain to sizing and targeting templates for use with bone plates having a twisted geometry that would otherwise pose difficulties pertaining to the targeting of their fixation holes. The templates disclosed herein are configured to temporarily anchor to a bone plate. The templates disclosed herein have features that, when anchored, identify target incision locations in the soft tissue, based on the respective soft tissue depths at the fixation holes, for targeting insertion of percutaneous instruments through the soft tissue and into the fixation holes. In at least some of the embodiments herein, the templates includes aperture arrangements having scales or gauges that correlate with the depth of the underlying tissue and helps the surgeon identify an associated target incision location for targeting the underlying fixation hole. These features provide the templates herein with low-cost solutions for increasing targeting efficiency for twisted bone plates.

As used in the specification including the appended claims, the singular forms “a,” “an,” and “the” include the plural, and reference to a particular numerical value includes at least that particular value, unless the context clearly dictates otherwise.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The terms “approximately”, “about”, and “substantially”, as used herein with respect to dimensions, angles, ratios, and other geometries, takes into account manufacturing tolerances. Further, the terms “approximately”, “about”, and “substantially” can include 10% greater than or less than the stated dimension, ratio, or angle. Further, the terms “approximately”, “about”, and “substantially” can equally apply to the specific value stated.

It should be understood that, although terms involving numerical prepositions (e.g., “first,” “second,” “third”) can be used herein to describe various features, such features should not be limited by these terms. These terms are instead used to distinguish one feature from another. For example, a first element could be termed a second element in another context, and, similarly, a second element could be termed a first element in another context, without departing from the scope of the embodiments disclosed herein.

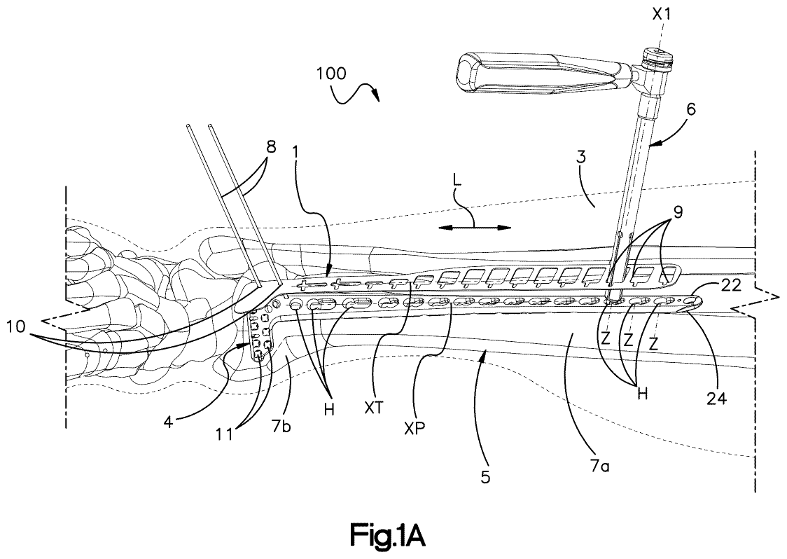

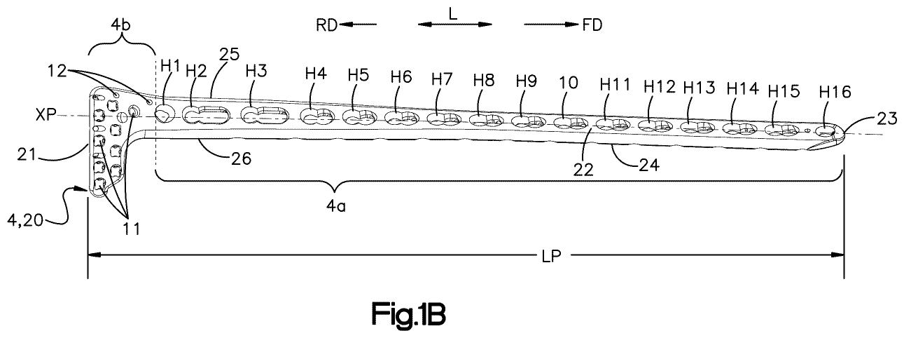

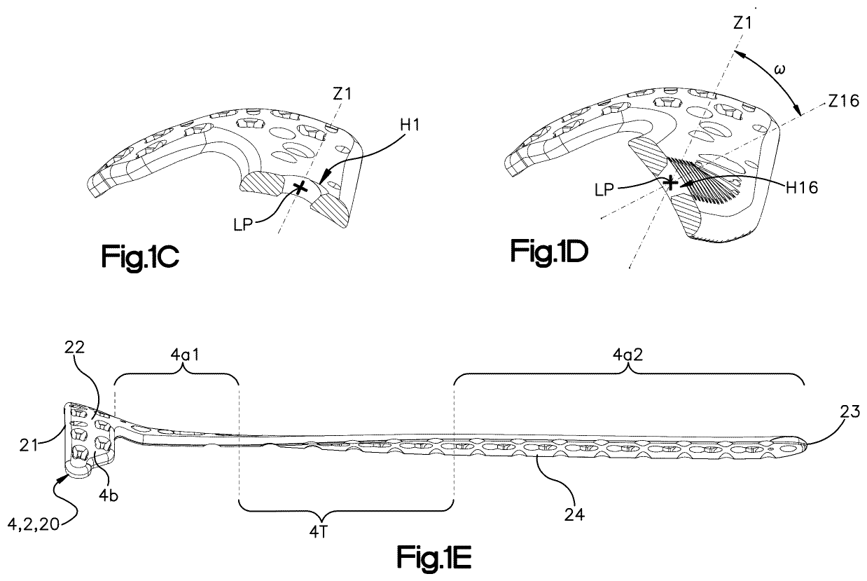

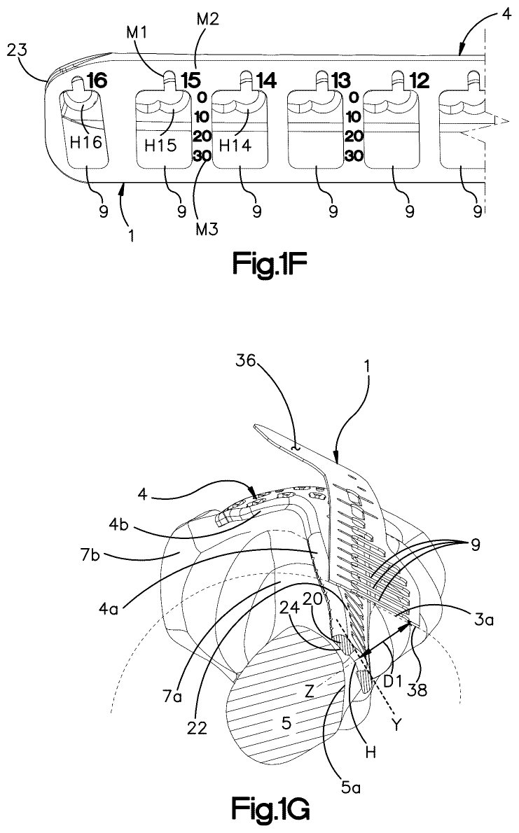

Referring now to FIGS. 1A-1E, an exemplary surgical system 100 is shown for guiding instrumentation through soft tissue 3 to respective features of a bone plate 4 that interfaces with underlying bone 5. The surgical system 100 includes a sizing and targeting template body 1 (also referred to herein as a “template” 1), a bone plate 4, one or more guide sleeves 6 for extending through the template 1 along an insertion trajectory X1 to target select fixation holes H of the bone plate 4, and one or more anchors 8 for affixing the template 1 to the bone plate 4. The bone plate 4 has a plate body 20 elongate along a longitudinal plate axis XP, which is preferably substantially parallel with the anatomical axis of the bone 5.

As shown in FIG. 1B, the plate body 20 has a first end 21 and a second end 23 opposite each other along the longitudinal plate axis XP. The plate body 20 also has a first side 25 and a second side 26 opposite each other along a direction substantially perpendicular to the longitudinal plate axis XP. The bone plate 4 of the illustrated embodiments is configured to temporarily (i.e., intra-operatively) anchor with the template 1 and with the underlying bone 5 (via the one or more anchors 8, as shown in FIG. 1A) at a location near the first end 21 and remote from the second end 23 of the plate body 20. Accordingly, for purposes of this disclosure, the first end 21 can be characterized as an “anchored end” 21 of the plate body 20, and the second end 23 can be characterized as a “free end” 23 of the plate body 20.

The plate body 20 also defines an outer plate surface 22 configured to face away from the underlying bone 5 and a bone-facing surface 24 configured to face the underlying bone 5. The outer surface 22 and the bone-facing surface 24 preferably extend substantially parallel to each other between the first and second ends 21, 23 of the plate body 20. The plate body 20 defines one or more fixation holes H that extend from the outer plate surface 22 to the bone-facing surface 24 along one or more respective central hole axes Z. The fixation holes H are configured for receiving respective bone fixation members (e.g., bone screws) for durably affixing the bone plate 4 to the underlying bone 5.

The bone plate 4 has a three-dimensional (3D) geometry that is configured to interface, preferably in complementary fashion, with an outer surface 5a of the underlying bone 5. As shown in FIGS. 1A and 1E, the template 1 is configured for placement on an outer surface 3a (e.g., skin surface 3a) of the soft tissue 3 overlaying the bone plate 5, particularly for guiding insertion trajectories X1 of instrumentation through the soft tissue 3 and to an associated fixation hole H. The template 1 is elongate along a longitudinal template axis XT oriented along a longitudinal direction L. It should be appreciated that the template 1 and bone plate 4 are configured such that the longitudinal template axis XT and the longitudinal plate axis XP are substantially parallel with each other when the template 1 is anchored to the bone plate 4. Accordingly, the longitudinal direction L is used herein synonymously with reference to the template 1 and the bone plate 4, based on their preferred relative position to each other.

In the illustrated embodiments herein, the bone plate 4 is a tibial plate, particularly for affixing to an anterior, distal region of a tibia 5. Accordingly, the illustrated template 1 is configured for placement on the skin 3a of the shin and ankle. It should be appreciated, however, that the bone plate 4 and templates 1 herein can be adapted for plate fixation to various other bones, including the fibula, femur, humerus, radius, ulna, scapula, clavicle, sternum, and ribs, by way of non-limiting examples.

As shown in FIGS. 1A and 1D-1E, the template 1 has a plurality of aperture arrangements 9 that align with associated fixation holes H of the bone plate 4 when the template 1 is anchored to the plate 4. The aperture arrangements 9 are configured to assist a surgeon in the task of orienting insertion trajectories X1 of bone screws through the soft tissue 3 and through associated fixation holes H in the underlying bone plate 4, as described in more detail below. The template 1 preferably includes visual indicia that indicates various information pertaining to the aperture arrangements 9 and associated fixation holes H. Such visual indicia includes various markers, preferably including, for example, hole markers M1 that indicate the locations of respective fixation holes H in the bone plate 4 associated with respective aperture arrangements 9 of the template 1. The visual indicia also preferably includes hole identifiers or “call-outs” M2 that identify the specific fixation holes H (in the series) associated with the aperture arrangements 9. The visual indicia also preferably includes a scale or gauge of secondary markers M3 (also referred to herein as a “depth gauge” M3) that correlates with respective tissue depths D1 associated with the respective fixation holes H, as described in more detail below.

As shown, the one or more anchors 8 can include a pair of K-wires 8 configured to extend through a pair of anchor holes 10 in the template 1 and through an associated pair of anchor holes 12 (FIG. 1B) in the bone plate 4 to substantially affix a relative position between the template 1 and the bone plate 4, thereby substantially maintaining longitudinal alignment of the aperture arrangements 9 with the associated fixation holes H. It should be appreciated, however, that other anchor configurations can be employed for affixing the template 1 to the bone plate 4 in such manner, such as other fixation wires, pins, nails, screws, and the like.

As shown in FIGS. 1A and 1E, the exemplary bone plate 4 has an elongated shaft region 4a for interfacing with the tibial shaft 7a and an end region 4b for interfacing with the malleolar region 7b of the distal tibia 5. The fixation holes H can be positioned in a longitudinal series along the shaft plate region 4a. The end plate region 4b preferably includes additional fixation holes 11 for affixing the end plate region 4b with the malleolar region 7b of the distal tibia.

Referring again to FIG. 1B, the bone plate 4 has a plate length LP measured from the first plate end 21 to the second plate end 23 along the longitudinal direction L. In plating procedures, the selected size of the bone plate 4 (particularly the plate length LP) can effectively determine the number of fixation holes H along the shaft plate region 4a. Or, stated differently, the plate length LP can be selected based on the number of fixation holes H desired along the shaft plate region 4a for a particular plating procedure. In plating procedures involving fixation to an end region of a long-bone (such as the malleolar region 7b of the distal tibia 5), variance in the selected plate length LP typically involves variance in the distance from which the plate 4 extends away from the malleolar region. Thus, for purposes of this disclosure, the series of fixation holes H preferably begins adjacent the first end 21 of the bone plate 4 and terminates adjacent the second end 23 of the bone plate 4. Thus, the fixation holes H in the series can be said to ascend in number in a forward direction FD extending from the first end 21 to the second end 23. Accordingly, the fixation holes H in the series can also be said to descend in number in a rearward direction RD opposite the forward direction FD. It should be appreciated that the forward and rearward directions FD, RD of this disclosure are each mono-directional components of the longitudinal direction L, which is bi-directional. Accordingly, the forward and rearward directions FD, RD can be used herein synonymously with reference to the template 1 and the bone plate 4, based on the preferred template-plate relative position.

In the illustrated embodiments herein, the shaft plate region 4a has a series of sixteen (16) fixation holes H1-H16 (FIG. 1B) spaced at longitudinal intervals along the longitudinal plate axis XP. In the series, the first fixation hole H1 is the hole of the shaft plate region 4a located nearest the first plate end 21, and the final fixation hole (hole H16 in the illustrated example) is located nearest the second, free plate end 23. It should be appreciated that in other embodiments the bone plate 4 can have more than or fewer than sixteen (16) fixation holes H. It should also be appreciated that the intervals between the holes H can be regular (i.e., equivalent) intervals, irregular intervals, or a combination of regular and irregular intervals.

Furthermore, in other embodiments, one or more and up to all of the fixation holes H can be eccentrically positioned with respect to the longitudinal plate axis XP (i.e., the central hole axis/axes Z of such fixation hole(s) H can be laterally offset from the longitudinal plate axis XP). One or more of the fixation holes H can be locking holes, compression holes, or combination holes (“combi-holes”) having a locking hole portion that intersects a compression hole portion. It should be appreciated that the bone plates 4 can have various fixation hole configurations, which can be accommodated and targeted by the aperture arrangements 9 of the templates 1 disclosed herein.

Because the bone plate 4 has a geometry that is complementary with the underlying tibia 5, the outer and bone-facing plate surfaces 22, 24 can have a twisted 3D geometry that is complementary with the contour of the underlying outer surfaces 5a of the tibia 5. The twisted plate geometry involves twist about the longitudinal plate axis LP. Referring now to FIGS. 1C-1D, an example of the extent or degree of plate twist between respective fixation holes H is shown. For purposes of this disclosure, the degree of plate twist between respective fixation holes H can be referenced by comparing the angular orientations of the respective central hole axes Z about a common axis, such as the longitudinal plate axis LP. In the illustrated example, the degree of plate twist is shown between the first fixation hole H1 and the sixteenth fixation hole H16 in the series. The longitudinal plate axis LP is shown intersecting both of the respective hole axes Z1, Z16 at the respective geometric centers of the fixation holes H1, H16. The degree of plate twist can be measured as the angle ω (referred to herein as the “twist angle” ω) between the respective hole axes Z1, Z16 about the common axis (e.g., longitudinal plate axis LP). In this manner, the twist angle @ between any respective pair of the fixation holes H can be measured. In the illustrated example, the twist angle ω between the first and last holes (i.e., holes H1 and H16) is about 45-degrees, although it should be appreciated that that the twist angle ω between the first and last fixation holes H in the series can be less than or greater than 45-degrees.

Referring now to FIG. 1E, a majority and up to an entirely of the plate twist can occur along a “twist portion” 4T of the shaft plate region 4a. In the illustrated example, most of the plate twist occurs in the twist portion 4T, which is positioned longitudinally between first and second portions 4a1, 4a2 of the shaft region 4a. The first portion 4al of the shaft region 4a is positioned adjacent the first plate end 21, while the second portion 4a2 extends in the forward direction FD to the second plate end 23. It should be appreciated that, in this example, the first and/or the second portion 4a1, 4a2 of the shaft region 4a can also possess a minor degree of twist. As used herein with reference to a bone plate, the term “twisted portion” refers to any portion of the shaft region 4a along which any fixation hole H thereof has a central hole axis Z that is angularly offset at a twist angle o from the central hole axis Z1 of the first hole H1 about the longitudinal plate axis LP. It should be appreciated that the outer and bone-facing plate surfaces 22, 24 preferably maintain a substantially parallel relationship with each other along the twisted portions of the bone plate 4, including the twist portion 4T.

Twisted plate geometries, such as those disclosed herein, present challenges for targeting the fixation holes H, particularly when targeting the fixation holes H through interposed soft tissue 3, such as during a post-operative procedure or otherwise without the benefit of a retracted longitudinal incision through which the plate 4 was implanted. Such twisted plate geometry is particularly challenging when the twisted plate surfaces are non-complementary with the outer surface 3a (e.g., skin surface 3a) of the interposed soft tissue 3. Such plate geometries are challenging because, even when factors such as the angular offset between the skin surface 3a and the outer plate surface 22 at the target site (i.e., at a fixation hole H) are generally known (such as via X-ray imagery), additional factors are necessary to identify the desired incision location on the skin surface 3a to target the hole H. One such factor, that is critically employed by the templates 1 herein, is the target tissue depth D1 measured from the skin surface 3a to a target of the insertion trajectory X1, preferably to the outer plate surface 22 at the target fixation hole H. More specifically, the target tissue depth D1 is preferably measured to the outer opening of the fixation hole H, i.e., the location where the measurement would encounter the outer plate surface 22 if it extended uninterrupted over the fixation hole H, which location is indicated by axis Y in FIG. 1G. In other embodiments, the templates 1 can be adapted for a target tissue depth D1 measured to the bone surface 5a through the target hole H. As shown in FIG. 1C, the target tissue depth D1 is preferably measured along the central hole axis Z of the targeted fixation hole H. The templates 1 herein are configured to target the fixation holes H substantially at a nominal screw insertion angle, although some marginal degree of screw angulation can be expected. It should be appreciated that the target tissue depth D1 can be measured via X-ray imagery, other types of medial imagery, such as ultrasound or MRI imagery. Additionally or alternatively, the target tissue depth DI can be measured via instrumentation, such as scale or gauge presented on an outer surface of an instrument, such as on the outer surface of the guide sleeve 6, by way of a non-limiting example.

The templates 1 disclosed herein include features for utilizing the measured target tissue depths D1 associated with the fixation holes H to identify target incision locations on the skin surface 3a.

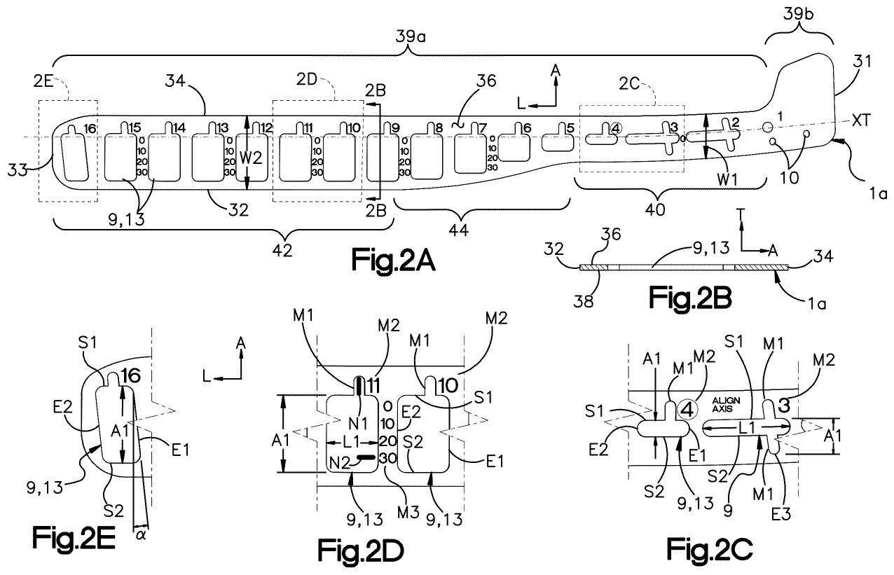

Referring now to FIGS. 2A, 3A, and 4A, exemplary templates 1a-c of the present disclosure each have a first end 31 and a second end 33 opposite each other along the longitudinal direction L. The first end 31 and second ends 33 of each template 1a-c are associated with the first and second ends 21, 23 of the bone plate 4. Accordingly, for purposes of this disclosure, the first and second ends 31, 33 of the templates 1a-c can be characterized as the “anchored end” 31 and the “free end” 33, respectively, of the templates 1a-c. Each template 1a-c also has a first side 32 and a second side 34 opposite each other along a lateral direction A substantially perpendicular to the longitudinal direction L. Each template 1a-c includes an outer surface 36 and a bone-facing surface 38 opposite each other along a transverse direction T substantially perpendicular to the longitudinal and lateral directions L, A. It should be appreciated that, as used herein; the terms “longitudinal”, “longitudinally”, and derivatives thereof refer to the longitudinal direction L; the terms “lateral”, “laterally”, and derivatives thereof refer to the lateral direction A; and the terms “transverse”, “transversely”, and derivatives thereof refer to the transverse direction T.

Each template 1a-c has a main template portion 39a that is configured to overlay the shaft portion 4a of the bone plate 4. The main template portion 39a defines at least some and preferably all of the aperture arrangements 9, which are spaced from each other along the longitudinal direction L. Each aperture arrangement 9 is associated with one or more respective fixation holes H of the bone plate 4. For example, an aperture arrangement 9 can be associated with a single one of the fixation holes H, on a one-to-one basis, or can be associated with multiple fixation holes H in a grouped basis.

Each template 1a-c also preferably has an anchored end portion 39b that extends from the main template portion 39a to the first end 31 in the rearward direction RD. The anchored end portion 39b preferably has a lateral geometry that generally corresponds to that of the end region 4b of the bone plate 4. As shown, the anchor holes 10 of the templates 1a-c can be located in the anchored end portion 39b.

To account for the increasing twist of the bone plate 4 moving from the anchored end 21 to the free end 23 of the plate 4 (i.e., moving in the forward direction FD), at least respective portions of the templates 1a-c preferably increase in lateral width moving along the template 1a-c from the anchored end 31 to the free end 33 thereof in the forward direction FD. For example, the shaft portion 39a of the templates 1a-c can have a first shaft portion 40 adjacent the anchored end portion 40b, and a second shaft portion 42 that is forwardly spaced from the first shaft portion 40 and extends forwardly toward the free end 33. Preferably, the second shaft portion 42 has a second lateral width W2 that is greater than a first lateral width W1 of the first portion 40, as measured laterally between the first and second sides 32, 34 of the template 1a-c. The increased lateral width W2 of the second portion 42 provides the aperture arrangements 9 thereof with increased lateral space to account for the plate twist and variance in the target tissue depth D1, as described in more detail below. The templates 1a-c can also have a transition portion 44 that extends between the first and second shaft portions 40, 42 and that widens laterally from the first lateral width W1 to the second lateral width W2, moving in the forward direction FD. Alternatively, the first and second portions 40, 42 can have substantially equivalent lateral widths (thereby omitting the transition portion 44), which can correspond to the increased lateral widths W2 shown in FIGS. 2A, 3A, and 4A.

Each of the aperture arrangements 9 has one or more apertures 13 extending from the outer surface 36 to the bone-facing surface 38 (FIG. 2B) along the transverse direction T. For example, each aperture arrangement 9 has at least one aperture 13, and optionally multiple apertures 13 arranged in various configurations. Each aperture arrangement 9, whether comprising a single aperture 13 of multiple apertures 13, defines a maximum lateral dimension A1 that correlates with the degree of twist in the geometry of the underlying portion of the bone plate 4. Accordingly, the maximum lateral dimension Al can increase from at least one of the aperture arrangements 9 to a subsequent one of the aperture arrangements 9 in the forward direction FD. For example, the maximum lateral dimension Al can increase by a factor of at least 1.5 from at least one of the aperture arrangements 9 (such as a first aperture arrangement 9 adjacent the first end 31) to at least another one of the aperture arrangements 9 (such as a final aperture arrangement 9 adjacent the second, free end 33). In further embodiments, the maximum lateral dimension A1 can increase by a factor of at least 2.0 from at least one of the aperture arrangements 9 to at least another one of the aperture arrangements 9 of the template 1.

The maximum lateral dimension Al is sufficient to allow an insertion trajectory X1 to intersect both 1) an aperture 13 of the aperture arrangement 9, and 2) an associated fixation hole H in a twisted portion of the underlying bone plate 4, at least within a range of target insertion depths D1. Thus, it can be said that at least a subset of the apertures 13 (i.e., one or more of the apertures 13) of each template la-c are sized and positioned such that, when the template 1a-c is anchored to and overlays the bone plate 4, at least one straight insertion trajectory X1 is orientable so as to intersect one of the apertures 13 and an associated fixation hole H extending through a twisted portion of the bone plate 4. Preferably, a subset of apertures 13 are sized and positioned such that multiple straight insertion trajectories X1 are orientable to intersect, respectively, multiple of the apertures 13 and associated fixation holes H extending through twisted portions of the bone plate 4.

As mentioned above, the aperture arrangements 9 of each template la-c preferably have associated visual indicia that indicates various information pertaining to the aperture arrangements 9 and associated fixation holes H. For example, at least some of (and preferably all of) the aperture arrangements 9 include at least one hole marker that indicates the location of at least one respective, underlying fixation hole H in the bone plate 4. The at least one hole marker can include a structural formation M1, such as a marking notch M1, that intersects an aperture 13 of the aperture arrangement 9. In such embodiments, the marking notch M1 preferably aligns with a center of the fixation hole H along the lateral direction A. The marking notch M1 extends from the outer surface 36 to the bone-facing surface 38 of the template 1a-c.

Additionally or alternatively, the at least one hole marker can include a mark, such as a tick mark, positioned to indicate the location of the underlying fixation hole H. Additionally or alternatively, the at least one hole marker can also include hole identifiers M2 having characters or numerals presented on the outer surface 36 of the template 1a-c at a position to indicate the location of the underlying fixation hole H. For example, the at least one hole marker can include a marking notch M1 positioned over a portion of the underlying fixation hole H, and can also include a hole identifier M2 (e.g., numeral(s)) adjacent the marking notch M1. Preferably, the template 1a-c has at least one hole marker for each of the fixation holes H of the shaft portion 4a of the bone plate 4.

One or more and up to each of the aperture arrangements 9 can include a depth gauge M3 associated with respective target tissue depths D1 at the underlying fixation hole(s) H. The depth gauge M3 is preferably arranged in rows spaced from each other along the lateral direction A. The depth gauge M3 can include numerals, which can indicate lateral positions of the aperture arrangement 9 associated with respective target tissue depths D1. For example, the depth gauge M3 can include the numerals “0”, “10”, “20”, and “30” arranged in respective rows and indicating the respective target tissue depths D1 in millimeters (mm), as associated with the respective lateral locations of the rows. In other embodiments, the numerals of the depth gauge M3 can indicate the respective target tissue depths D1 in other units, such as centimeters (cm) or inches (in.).

Referring now to FIGS. 2A-2E, a first exemplary embodiment of a template 1a will now be described. In this exemplary embodiment, at least a majority of the aperture arrangements 9 respectively consist of a single aperture 13 having a substantially rectangular, window-like configuration that overlays the respective fixation hole H. The window-like single-apertures 13 and sized to receive a percutaneous inserter assembly, which can include a guide sleeve 6 as shown in FIG. 1A. The template la of this embodiment can be referred to herein as a “window template” 1a. The apertures 13 extend longitudinally between first and second ends E1, E2 and extend laterally between first and second sides S1, S2. As shown, most of the apertures 13 of the present embodiment have a substantially rectangular shape, such as those apertures 13 associated with fixation holes H5-H16. Additionally, of those single-aperture arrangements 9, a majority of those along the second, wider portion 42 of the window template 1a have a maximum lateral dimension Al that is greater than a maximum longitudinal dimension L1 of the apertures 13, causing such apertures 13 to be elongate along the lateral direction A, as shown in FIG. 2D. Additionally, in the present embodiment, each aperture arrangement 9 is associated with a respective fixation hole H (i.e., on a one-to-one basis). The maximum lateral dimension A1 is preferably no less than 10 mm. More particularly, the maximum lateral dimension A1 and can be in a range from about 8 mm to about 30 mm, and more particularly in a range from about 10 mm to about 20 mm, and more particularly in a range of about 12 mm to about 16 mm.

Moreover, in the illustrated example of the window template la, each aperture arrangement 9 includes a marking notch M1 and hole identifier M2 for the associated fixation hole H. The marking notches M1 are positioned on the first sides S1 of the apertures 13. In particular, each marking notch M1 extends laterally away from the first side S1 of the respective aperture 13 toward the second side 34 of the window template 1a. Some of the aperture arrangements 9 also include a depth gauge M3 presented alongside the first or second end E1, E2 of the aperture 13. As shown, most of the aperture arrangements 9 along the second, wider portion 42 of the window template la are arranged in pairs, in which the depth gauge M3 is positioned between the second end E2 of one aperture 13 and the first end E1 of the next aperture 13 in the pair. Such pairs include the aperture arrangements 9 associated with fixation holes H6-H7, H8-H9, H10-H11, H12-H13, and H14-H15 in the illustrated embodiment. It should be appreciated that the visual indicia (hole markers M1, hole identifiers M2, and/or depth gauges M3) can be configured differently than that shown in FIGS. 2A-2E while remaining within the scope of the present disclosure.

As shown in FIG. 2E, in the final aperture arrangement 9 in the series (i.e., the aperture arrangement 9 closest to the free end 32 of the template la), end surfaces of the aperture 13 at the ends E1, E2 can be angled at an acute back angle α with respect to the lateral direction A.

As shown in FIG. 2C, one or more of the aperture arrangements 9 can include first and second marking notches M1 on opposite sides S1, S2 of the aperture 13. In particular, the first and second marking notches M1 extend laterally away from each other from the first and second sides S1, S2 of the aperture 13. In the illustrated embodiment, the aperture arrangements 9 with first and second opposed marking notches M1 are those associated with fixation holes H2 and H3, although other aperture arrangements 9 can employ first and second opposed marking notches M1.

In the present embodiment, the marking notches M1 are positioned longitudinally with respect to the first end El of the associated aperture 13 so as to align the guide sleeve 6 with the center of the fixation hole H. In particular, when an outer surface of the guide sleeve 6 contacts the first end El at a lateral position thereof associated with the target tissue depth D1 (as indicated by the scale of secondary markers M3), the guide sleeve 6 can pivot laterally (i.e., pivots about a pivot axis oriented along the longitudinal direction L) substantially without incurring a longitudinal offset between the central sleeve axis Y and the central hole axis Z. In this manner, the marking notch M1, the first end El of the aperture 13, and the scale of secondary markers M3 assist the surgeon with targeting the fixation hole H with the guide sleeve 6.

It should be appreciated that additional aspects of the surgical system 100 can assist the physician with orienting the guide sleeve 6 to target the fixation hole H. For example, the positions of the anchor holes 10, 12 in the window template 1a and bone plate 4, respectively, can be configured such that the anchors 8 extend therethrough at an orientation that approximates a median trajectory angle of the guide sleeve 6 (e.g., such as for a target tissue depth D1 of 20 mm) for one or more of the apertures 13 of the present embodiment. In the illustrated embodiment, for example, the anchors 8 are configured to extend through the respective templates 1a-c and the bone plate 4 at an orientation that is substantially parallel with the central hole axis Z4 of the fourth fixation hole H4. Accordingly, as shown in FIG. 2C, the template la can include alignment indicia adjacent the aperture 13 associated with the fourth fixation hole H4. The alignment indicia, e.g., “ALIGN AXIS”, can instruct the surgeon to employ visual reference to the anchors 8 to approximate the trajectory angle associated with the fourth fixation hole H4 and the respective aperture 13.

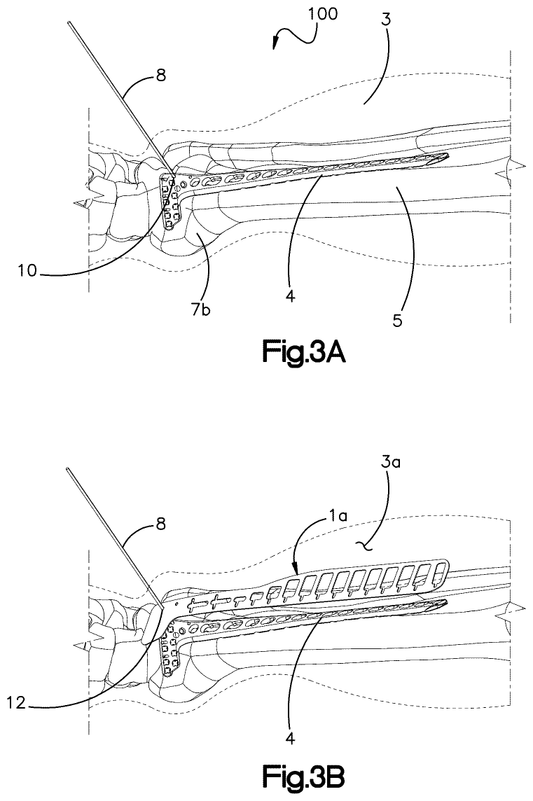

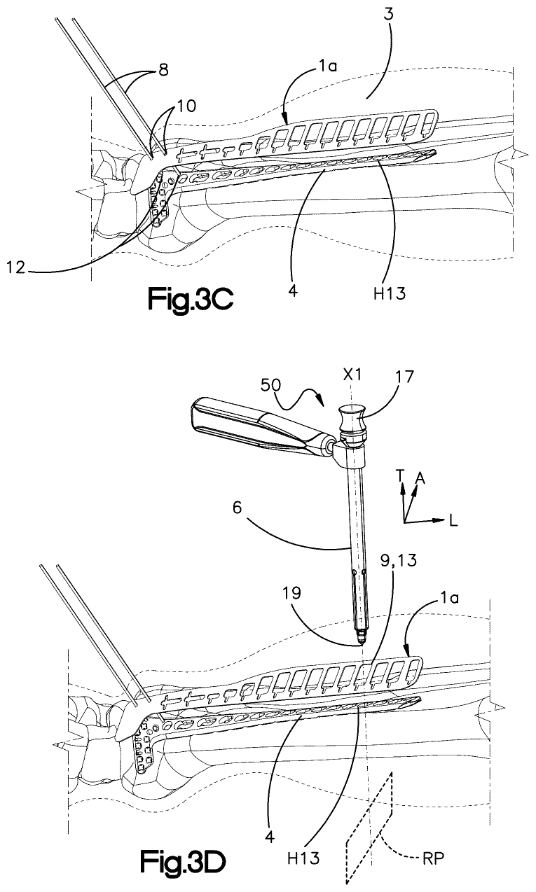

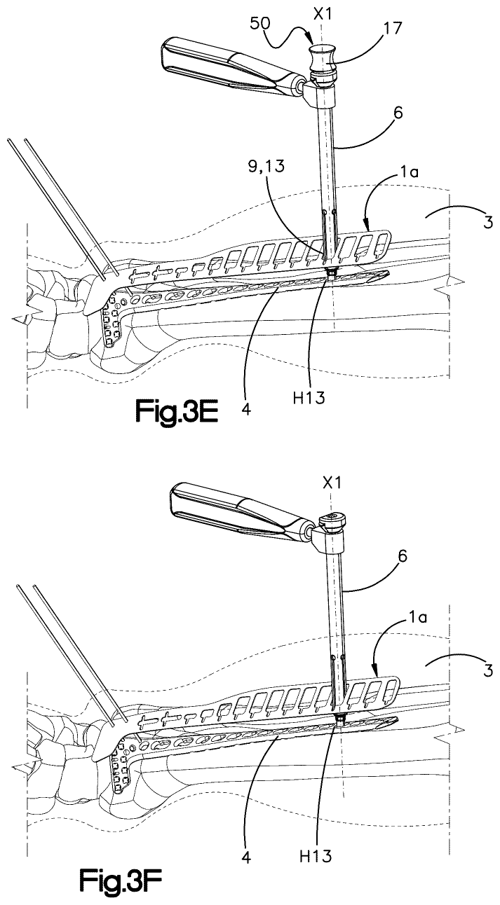

Referring now to FIGS. 3A-3F, an exemplary method of using the window template la during a bone plating procedure will now be described. Referring now to FIG. 3A, the bone plate 4 is shown placed adjacent underlying bone, which in this example is the patient's right tibia 5. An anchor member 8 (such as a guide wire, preferably a K-wire) is inserted through soft tissue 3 and through one of the anchor holes 12 of the bone plate 4 and into underlying bone 5, such as the malleolar region 7b of the tibia 5. Referring now to FIG. 3B, the template la is advanced along the anchor member 8 (with the anchor member 8 extending through the associated anchor hole 10 of the template 1a) until the bone-facing template surface 38 contacts the patient skin 3a overlaying the bone plate 4 (FIG. 1G).

Referring now to FIG. 3C, a second anchor member 8 is inserted through the additional anchor hole 10 of the template la, through the underlying soft tissue, through the additional anchor hole 12 in the bone plate 4, and into the underlying bone (e.g., in the malleolar region 7b of the tibia 5). With the template la anchored to the bone plate 4 via the anchor members 8, the template la and the bone plate 4 extend longitudinally in generally parallel fashion, with the bone plate 4 underlying the template la. At this stage, the template la can be employed for: (1) sizing the length LP of the bone plate 4; and (2) marking the location on the patient skin for each incision for percutaneous screw insertion.

During the sizing step, the surgeon correlates the known length LP of the bone plate 4 with the total number of fixation holes H in the series, which holes H are indicated on the template la by the hole markers M1 and hole identifiers M2. Before or during the marking step, target tissue depth D1 measurements are performed at the fixation holes H, particularly for those holes H along the twist portion 4T and second portion 4a2 of the bone plate 4. As discussed above, the target tissue depths D1 can be measured via X-ray imagery or other types of medial imagery.

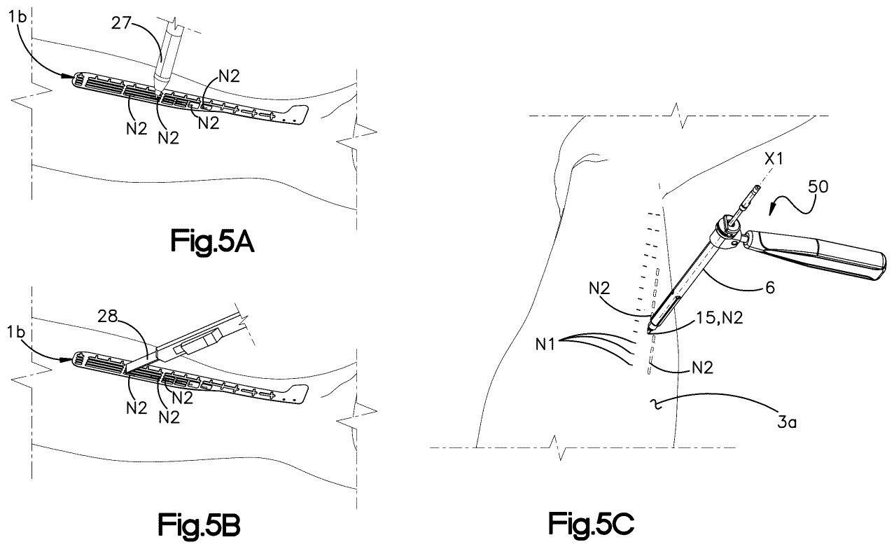

During the marking step, the surgeon can employ the template 1a to mark the patient skin at one or more locations associated with each of the respective holes H targeted for percutaneous screw insertion. Such marking can be made or “drawn” via a surgical marking pen 27 (see FIG. 5A) or other writing implement. The markings can include hole markings N1 (see FIG. 2D), which can be drawn on the skin through the marking notch M1 associated with each target hole H. The markings-particularly for each target hole H underlying the second and transition shaft portion 42, 44 of the template la-also preferably include incision markings N2 (see FIG. 2D), which are drawn through the associated aperture 13 at a lateral location thereof associated with the target tissue depth D1 as referenced to the depth gauge M3.

Additional steps of the exemplary method of using the window template 1a will be described with reference to targeting the thirteenth fixation hole H13 in the series of the illustrated embodiment of the bone plate 4. It should be appreciated, however, that the following steps can also apply to targeting any of the fifth through sixteenth fixation holes H5-H16 in the series, or targeting fixation holes of a different twisted bone plate.

Referring now to FIG. 3D, with the target tissue depth D1 measured at the target fixation hole H13, the surgeon can further employ the window template 1a to guide insertion of surgical instrumentation through the soft tissue 3 and to the target hole H13 of the bone plate 4. The window template la is particularly configured for use with a percutaneous inserter assembly 50, which can include a cannulated guide sleeve 6 for guiding various percutaneous instruments through the soft tissue 3 and to the target fixation hole H13. Such percutaneous instruments can include incision instruments, such as a soft tissue trocar 17, as shown, and/or a bone-hole opening tools, such as drill bits, by way of non-limiting examples. As shown, a trocar 17 can be inserted within, and fully seated with respect to, the guide sleeve 6 such that a distal cutting tip 19 of the trocar 17 extends distally from the guide sleeve 6.

With the trocar 17 coupled to the guide sleeve 6, the surgeon can bring the insertion assembly 50 into position adjacent the aperture arrangement 9 associated with the target fixation hole H13. During this step, the surgeon can refer to the hole marking N1 and incision marking N2 (see FIG. 2B) to identify the desired placement of the distal cutting tip 19 of the trocar 17. As discussed above, the window apertures 13 are sized such that the insertion trajectory X1 is longitudinally aligned with the associated fixation hole H13 when: (1) the outer surface of the guide sleeve 6 contacts the first end El of the aperture 13; and (2) the guide sleeve 6 is oriented such that the insertion trajectory X1 is coextensive with a reference plane RP oriented along the lateral and transverse directions A, T. Thus, when the surgeon places the guide sleeve 6 within the respective aperture 13 and in contact with the first end El thereof, specifically at the desired lateral position thereof (as indicated by the incision marking N2) and coextensive with the reference plane RP, the surgeon then need only pivot the guide sleeve 6 about a longitudinal pivot axis to orient the insertion trajectory X1 through the target hole H13 substantially at a nominal insertion angle. It should be appreciated that the foregoing pivoting step can be performed free-hand and/or with assistance of medical imagery.

Referring now to FIG. 3E, the insertion assembly 50 is shown having advanced through the select template aperture 13 along an insertion trajectory X1 that extends through the target fixation hole H13. During such advancement, the distal cutting tip 19 of the trocar 17 preferably creates a straight puncture incision through the soft tissue 3 until the distal end of the guide sleeve 6 seats within the fixation hole H13, or at least until the distal cutting tip 19 or the distal end of the guide sleeve 6 contacts the bone plate 4 in close proximity to the fixation hole H13. In the latter scenario, the surgeon can further manipulate the guide sleeve 6 using tactile feedback until the distal end of the guide sleeve 6 seats within the fixation hole H13.

Referring now to FIG. 3F, with the distal end of the guide sleeve 6 seated within the target fixation hole H13, the trocar 17 can be removed from the guide sleeve 6, and additional percutaneous instrumentation can be employed through the guide sleeve 6 to facilitate screw insertion within the hole H13 and into the underlying bone 5. For example, a drill bit can be advanced through the guide sleeve 6 and through the fixation hole H13 to pre-drill a hole in the underlying bone. Subsequently, the drill bit can be withdrawn and a bone screw and a driving tool can be inserted through the guide sleeve 6 for driving the bone screw through the fixation hole H13 and into the pre-drilled hole in the bone until a head of the bone screw fully seats within the fixation hole H13. After screw insertion, the insertion assembly 50 can be withdrawn from the soft tissue 3 and from the template la, and can then be employed in similar fashion repeating the foregoing steps for one or more additional target fixation holes H using the associated aperture arrangement 9 of the template la.

It should be appreciated that the foregoing method described above with reference to FIGS. 3A-3F can include additional and/or alternative steps while remaining within the scope of the present disclosure. It should also be appreciated that the sequence of various steps in the foregoing method can be adjusted as needed.

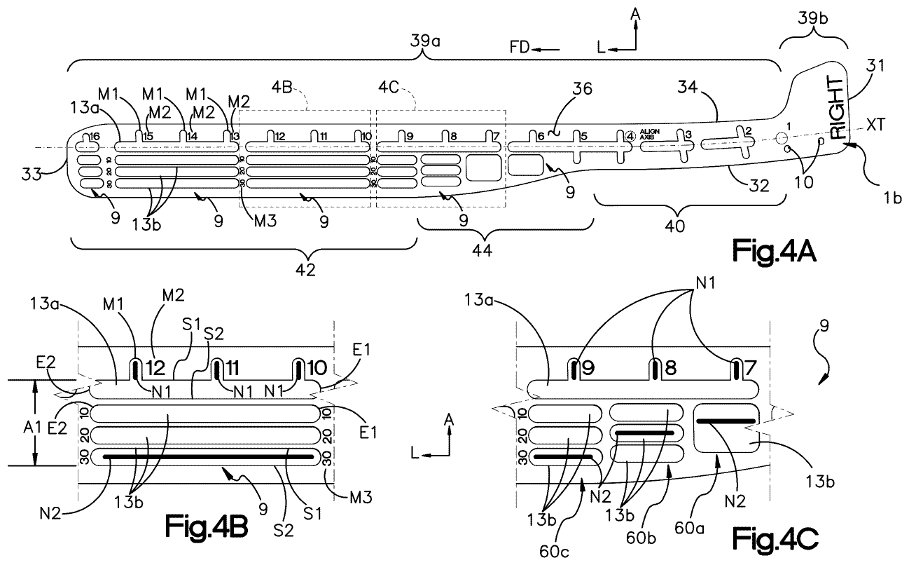

Referring now to FIGS. 4A-4C, a second exemplary embodiment of a template 1b will now be described, in which one or more of the aperture arrangements 9 have a plurality of longitudinally elongate apertures 13 (which can be referred to herein as “slots” 13) that are laterally spaced from each other. Accordingly, the template 1b of this embodiment can be referred to herein as a “slotted template” 1b. The slots 13 of the present embodiment are configured to assist the surgeon with drawing straight longitudinal markings on the patient's skin at longitudinal positions associated with target holes H and lateral positions correlated with the target tissue depths D1. For the sake of brevity, the following description will generally focus on aspects of the slotted template 1b that are different than those of the window template 1a described above. Accordingly, it should be appreciated that similar reference characters to those discussed above in connection with FIGS. 1A-3F can be used to indicate similar features of the slotted template 1b described in connection with FIGS. 4A-4C.

One difference between the window template 1a discussed above and the slotted template 1b it that, although the slotted template 1b can be employed for sizing and marking in generally similar fashion to the window template la, the slotted template 1b is configured to be removed from the patient prior to percutaneous screw insertion.

Additionally, in the slotted template 1b, one or more of the aperture arrangements 9 are individually associated with multiple fixation holes H. For example, most of the aperture arrangements 9 of the slotted template 1b are associated with groups of three (3) fixation holes H. Additionally, particularly along the second, wider portion 42 of the template 1b, some of the aperture arrangements 9 each have a first or “base” slot 13a and one or more secondary slots 13b that are each longitudinally elongate and are laterally spaced from each other. In such aperture arrangements 9, marking notches M1 for each of the associated fixation holes H extend from a first side S1 of the first slot 13a. Additionally, the secondary slots 13b are preferably aligned and associated with the markers of the depth gauge M3.

As shown in FIGS. 4A-4B, the first slot 13a can be aligned with the “0 ” (zero) position or numeral of the depth gauge M3. At such position, the first slot 13a can be employed to mark a target incision for one or more underlying fixation holes H having either no twist angle ω or de minimis twist angle ω. As shown, the secondary slots 13b can include two or more (e.g., three (3)) secondary slots 13b arranged in a lateral series spaced from the first slot 13a. The secondary slots 13b are preferably aligned with respective numerals (e.g., “10”, “20”, and “30” mm indicators) of the depth gauge M3. The maximum lateral dimension A1 for each respective aperture arrangement 9 is measured from the first side S1 of the first slot 13a to the most laterally spaced second side S2 of a slot 13a,b of the aperture arrangement 9. Accordingly, in the illustrated embodiment of the slotted template 1b, the maximum lateral dimensions A1 of the aperture arrangements 9 associated with the second and third fixation holes H2, H3 are measured from the first side S1 of the first (and only) slot 13a to the most laterally spaced end E3 of the second marking notch M1 (as similarly shown in FIG. 2C). However, for the aperture arrangements 9 associated with grouped fixation holes H7-H9, H10-H12, and H13-H15, and with fixation hole H16, the maximum lateral dimensions A1 for these respective aperture arrangements 9 are measured from the first side S1 of the first slot 13a to the second side S2 of the most laterally spaced secondary slot 13b.

As shown in FIGS. 4A and 4C, the secondary slots 13b of one or more of the aperture arrangements 9 can include two or more longitudinally spaced subsets of secondary slots 13b, such as three (3) subsets 60a-c of secondary slots 13b, wherein each subset 60a, 60b, 60c can be associated with a respective on of the fixation holes (e.g., H7, H8, and H9, respectively). Optionally, one or more of the subsets 60a-c can consist of a single, window-like secondary slot 13b, such as the subset 60a associated with fixation hole H7 in FIG. 4C.

As shown in FIGS. 4B-4C, the first slot 13a and secondary slots 13b of an aperture arrangement 9 can each be elongate in the longitudinal direction L. However, it should be appreciated that, in other embodiments, the first slot 13a and/or one or more of the secondary slots 13b of an aperture arrangement 9 can be elongate in a respective direction that is offset from the longitudinal direction L yet having at least a directional component in the longitudinal direction L.

With additional reference to FIGS. 5A-5C, an exemplary method of using the slotted template 1b during a bone plating procedure will now be described. It should be appreciated that the slotted template 1b can be anchored to the bone plate 4 and to the underlying bone (e.g., tibia) in the manner described above with reference to FIGS. 3A-3C. Moreover, the slotted template 1b can be employed for sizing the plate length LP (i.e., identifying the number of fixation holes H in the series) by using the marking notches M1 and hole identifiers M2 in similar fashion to that described above. Furthermore, the target tissue depth D1 measurements can be performed at the target fixation holes H, as also described above.

Referring now to FIG. 5A, the first slot 13a and secondary slots 13b of the slotted template 1b are particularly adapted for drawing target incision markings N2 on the patient's skin 3a, such as with a surgical marking pen 27 or other writing implement. In particular, the slots 13a,b are adapted to allow the surgeon to trace or otherwise draw the target incision markings N2 in the form of straight, longitudinal lines on the skin using specific slots 13a,b selected based on the target tissue depths D1 at the target fixation holes H. Additionally, as shown in FIG. 5B, the slots 13 a, b also provide the benefit of guiding movement of a scalpel 28 or other incising instrument to create respective incisions 15 at lateral locations correlated with the target tissue depth D1 and plate twist. It should be appreciated that the surgeon can elect to make the incisions while the template 1b is anchored to the patient or after the template 1b is removed. Referring now to FIG. 5C, after the hole markings N1 and target incision markings N2 have been drawn onto the skin 3a, the template 1b can be removed. After template removal, percutaneous screw insertion can be performed through the incisions 15. In the present example, the combination of the hole markings N1 and longitudinally extending target incision markings N2 provide useful references for the target tissue depth D1 (via the lateral spacing between the markings N1, N2), while the target incision markings N2 also provide a visual reference of a longitudinal pivot axis for pivoting the guide sleeve 6 to assist the surgeon with aligning the insertion trajectory X1 so that it extends through the target fixation hole H.

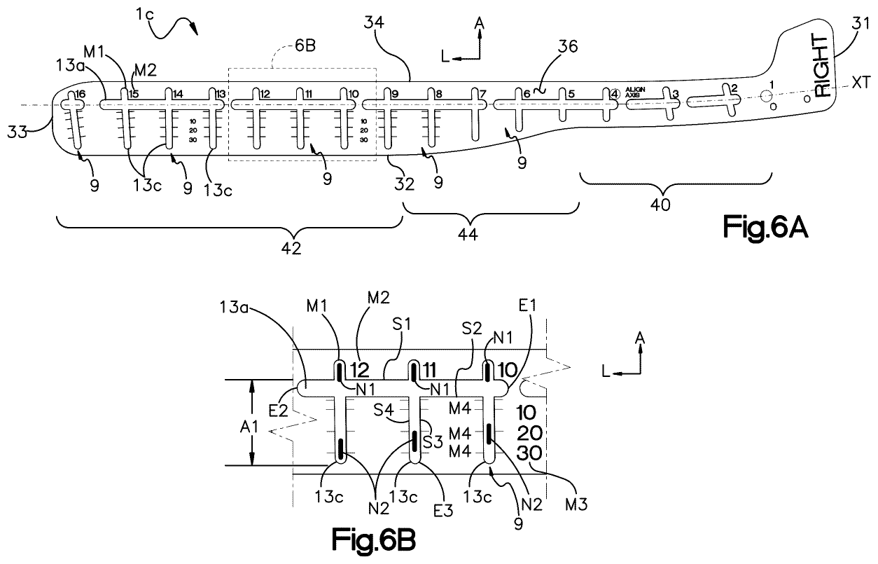

Referring now to FIGS. 6A-6B, a third exemplary embodiment of a template 1c will now be described. In the third exemplary template 1c, one or more of the aperture arrangements 9 includes a first slot 13a that is longitudinally elongate and at least one second slot 13c that intersects the first slot 13a and is laterally elongate. The laterally elongate second slots 13c facilitate laterally elongated target incision markings N2. Again for the sake of brevity, the following description will generally focus on aspects of the template 1c that are different than those of the window template la and the slotted template 1b described above. Accordingly, it should be appreciated that similar reference characters to those discussed above in connection with FIGS. 1A-5C can be used to indicate similar features of the slotted template 1b described in connection with FIGS. 4A-4C.

As shown in FIGS. 6A-6B, one or more of the aperture arrangements 9 can each include multiple second slots 13c that intersect the first slot 13a. In the illustrated example, such aperture arrangements 9 include three (3) second slots 13c, which extend laterally opposite three (3) respective marking notches M1. The visual indicia of the present embodiment can include tick marks M4 along the one or more sides S3, S4 of the second slots 13c. As shown, the tick marks M4 can be positioned in respective rows of the depth gauges M3.

It should be appreciated that the first slot 13a can be elongate along a direction that is offset from the longitudinal direction L yet has at least a directional component in the longitudinal direction L. Additionally or alternatively, the second slots 13b can be elongate along one or more respective directions that are offset from the lateral direction A yet each have at least a directional component in the lateral direction A.

It should also be appreciated that the third template 1c can be employed during a bone plating procedure for sizing the bone plate 4 and drawings the target incision markings in similar fashion to the exemplary method described above with respect to the slotted template 1b shown in FIGS. 4A-5C. One primary difference being that the target incision markings N2 facilitated by the third template 1c are laterally elongated as opposed to longitudinally elongated.

It should be appreciated that the various features of the templates 1a-c described above are provided as exemplary features for targeting fixation holes of a bone late through soft tissue. Aspects of these features can be adjusted as needed without departing from the scope of the present disclosure. For example, various aperture arrangements 9 of the respective embodiments above can be adapted based on the aperture arrangements of other embodiments disclosed above. Additionally, the size and geometry of the templates 1a-c and their respective aperture arrangements 9 can be adapted based on the types and sizes of associated bone plates for use therewith.

Although the disclosure has been described in detail, it should be understood that various changes, substitutions, and alterations can be made herein without departing from the spirit and scope of the invention as defined by the appended claims. Moreover, the scope of the present disclosure is not intended to be limited to the particular embodiments described in the specification. In particular, one or more of the features from the foregoing embodiments can be employed in other embodiments herein. As one of ordinary skill in the art will readily appreciate from that processes, machines, manufacture, composition of matter, means, methods, or steps, presently existing or later to be developed that perform substantially the same function or achieve substantially the same result as the corresponding embodiments described herein may be utilized according to the present disclosure.

What is claimed:

1. A surgical system for bone fixation comprises:
a bone plate for affixation to underlying bone, the bone plate comprising:
a first plate end and a second plate end opposite each other along a longitudinal plate axis;
an outer plate surface and an opposed bone-facing plate surface extending substantially parallel to each other between the first and second plate ends;
a plurality of fixation holes extending from the outer plate surface to the bone-facing plate surface, the plurality of fixation holes arranged in longitudinal series that begins adjacent the first plate end and terminates adjacent the second plate end; and
a twisted plate portion along which the outer and bone-facing plate surfaces have respective twisted geometries about the longitudinal plate axis; and
a template for placement on an outer surface of soft tissue overlaying the bone plate, the template comprising:
first and second template ends opposite each other along a longitudinal direction;
a plurality of apertures located between the first and second template ends, wherein the plurality of apertures are configured to orient insertion trajectories of bone screws through the soft tissue and through associated fixation holes in the bone plate,
wherein at least a subset of the plurality of apertures are sized and positioned such that, when the template overlays the bone plate, at least one of the insertion trajectories intersects:
an aperture of the subset of apertures, and
an associated fixation hole extending through the twisted plate portion.

2. The surgical system of claim 1, wherein the at least the subset of the plurality of apertures are sized and positioned such that, when the template overlays the bone plate, a plurality of the insertion trajectories intersect respective:
pluralities of the subset of apertures, and
associated fixation holes extending through the twisted plate portion.

3. The surgical system of claim 1, wherein the bone plate comprises a first plate portion positioned longitudinally between the first plate end and the twisted plate portion, wherein the first plate portion has less twist about the longitudinal plate axis than the twisted plate portion, and at least another subset of apertures of the template are sized and positioned such that, when the template overlays the bone plate, at least another one of the insertion trajectories intersects:
an aperture of the another subset of apertures, and
an associated fixation hole extending through the first plate portion.

4. The surgical system of claim 3, wherein the bone plate comprises a second plate portion positioned longitudinally between the twisted plate portion and the second plate end, wherein the second plate portion has less twist about the longitudinal plate axis than the twisted plate portion, and at least an additional subset of apertures of the template are sized and positioned such that, when the template overlays the bone plate, at least an additional one of the insertion trajectories intersects:
an aperture of the additional subset of apertures, and
an associated fixation hole extending through the second plate portion.

5. The surgical system of claim 1, wherein:
the second template end is spaced from the first template end in a forward direction along the longitudinal direction;
the template has first and second template sides opposite each other along a lateral direction substantially perpendicular to the longitudinal direction;
the subset of the plurality of apertures are grouped in respective aperture arrangements that are spaced from each other along the longitudinal direction, each aperture arrangement having at least one aperture of the plurality of apertures,
in each aperture arrangement, the respective at least one aperture defines a maximum lateral dimension along the lateral direction, and
the maximum lateral dimension increases from at least one of the aperture arrangements to a subsequent one of the aperture arrangements in the forward direction.

6. The surgical system of claim 5, wherein the maximum lateral dimension increases by a factor of at least 2.0 from the at least one of the aperture arrangements to the subsequent one of the aperture arrangements.

7. The surgical system of claim 5, wherein the template comprises:
a first template portion that defines the at least one of the aperture arrangements; and
a second template portion that is spaced from the first template portion in the forward direction and defines the subsequent one of the aperture arrangements,
wherein the template defines a lateral template width measured between the first and second template sides along the lateral direction, and the lateral template width along the second template portion is greater than the lateral template width along the first template portion.

8. The surgical system of claim 5, wherein at least one of the aperture arrangements comprises two or more apertures that are spaced from each other along one of the lateral and longitudinal directions.

9. The surgical system of claim 5, wherein at least a majority of the aperture arrangements respectively consist of a single aperture that is elongate along the lateral direction.

10. The surgical system of claim 1, wherein:
the bone plate comprises plate anchoring holes spaced from each of the plurality of fixation holes;
the template comprises template anchoring holes spaced from each of the plurality of apertures; and
the surgical system comprises anchor members configured to extend, respectively, through the template anchoring holes and the plate anchoring holes for substantially affixing a relative position between the template and the bone plate.

11. A template for placement on an outer surface of tissue overlaying a bone plate having a twisted geometry, the template configured to guide insertion trajectories of bone screws through the tissue and through respective fixation holes in the bone plate, the template comprising:
first and second ends opposite each other along a longitudinal direction, wherein the second end is spaced from the first end in a forward direction along the longitudinal direction;
a first side and a second side opposite each other along a lateral direction substantially perpendicular to the longitudinal direction;
an outer surface and a bone-facing surface opposite each other along a third direction substantially perpendicular to the longitudinal and lateral directions; and
a plurality of aperture arrangements spaced from each other along the longitudinal direction,
wherein at least some of the aperture arrangements each include:
one or more apertures that are elongated along the longitudinal direction and extend from the outer surface to the bone-facing surface along the third direction; and
at least one hole marker indicating a location of at least one respective, underlying fixation hole in the bone plate,
wherein a totality of the one or more apertures of the respective aperture arrangement defines a maximum lateral dimension along the lateral direction, and
wherein the maximum lateral dimension increases from at least one of the aperture arrangements to a subsequent one of the aperture arrangements in the forward direction.

12. The template of claim 11, wherein the maximum lateral dimension increases by a factor of at least 1.5 from the at least one of the aperture arrangements to the subsequent one of the aperture arrangements.

13. The template of claim 12, wherein the maximum lateral dimension increases by a factor of at least 2.0 from the at least one of the aperture arrangements to the subsequent one of the aperture arrangements.

14. The template of claim 11, wherein the maximum lateral dimension of the subsequent one of the aperture arrangements is at least 13.5 mm.

15. The template of claim 11, wherein the at least one hole marker includes:
a notch extending from at least one of the one or more apertures along the lateral direction; and
a numeral adjacent the notch, wherein the numeral identifies a respective fixation hole of the plurality of fixation holes.

16. The template of claim 11, wherein:
the at least some of the aperture arrangements include a scale of secondary markers associated with respective depths between the outer surface of the tissue and an outer surface of the bone plate at the at least one respective, underlying fixation hole;
the secondary markers of the scale of secondary markers are arranged in rows that are spaced from each other along the lateral direction; and
the scale of secondary markers includes numerals positioned in the rows, wherein the numerals indicate the associated respective depths in millimeters (mm).

17. The template of claim 1, wherein the one or more apertures comprise:
a first slot that is elongate in a first direction having at least a directional component along the longitudinal direction; and
at least one second slot elongate in a second direction having at least a directional component along the lateral direction, wherein the at least one second slot intersects the first slot.

18. The template of claim 1, wherein the one or more apertures comprise:
a first slot that is elongate in a first direction having at least a directional component along the longitudinal direction, the first slot having first and second sides opposite each other along the lateral direction, wherein the at least one hole marker is positioned on the first side of the first slot; and
a plurality of secondary slots spaced between the second side of the first slot and the second side of the template along the lateral direction, wherein the secondary slots are elongate in the first direction, and the secondary slots are each associated with a respective depth measured between the outer surface of the tissue and an outer surface of the bone plate at the at least one respective, underlying fixation hole.

19. The template of claim 1, wherein the at least some of the aperture arrangements each consist of a single aperture that extends between first and second aperture sides opposite each other along the lateral direction, and the maximum lateral dimension is defined from the first aperture side to the second aperture side.

20. The template of claim 19, wherein:
the single aperture is associated with a single one of the underlying fixation holes;
the single aperture is sized to receive a cannulated guide sleeve that defines a respective one of the insertion trajectories for a respective bone screw; and
the single aperture is additionally sized such that, when the template overlays the bone plate, the respective one of the insertion trajectories intersects both of the single aperture and the associated underlying fixation hole.
